(19) United States
(12) Patent Application Publication
(10) Pub. No.: US 2026/0297224 A1
(43) Pub. Date: Oct. 1, 2026

(54) PROCESS FOR ACTIVATING METALLOCENE CATALYSTS IN REACTOR TO INCREASE CATALYST PRODUCTIVITY IN PAO PRODUCTION

(71) Applicant: ExxonMobil Technology and Engineering Company, Spring, TX (US)

(72) Inventors: Andrew E. Atalla, Houston, TX (US); Patrick C. Chen, Houston, TX (US); Jennifer L. Rapp, Houston, TX (US)

(21) Appl. No.: 19/477,246

(22) PCT Filed: May 20, 2024
(86) PCT No.: PCT/US2024/030204

§ 371 Date: Oct. 21, 2025

Related U.S. Application Data
(60) Provisional application No. 63/503,554, filed on May 22, 2023.

(51) Int. Cl. C08F 10/14

(52) U.S. Cl. CPC C08F 10/14

(57) ABSTRACT

Processes for making poly alpha-olefins (PAO) at high catalyst efficiency, good kinetics, and high conversions are provided. In at least one embodiment, the process includes feeding at least one catalyst to an oligomerization reactor; feeding at least one activator to the oligomerization reactor; feeding one or more linear alpha olefins to the oligomerization reactor and oligomerizing the one or more linear alpha olefins within the oligomerization reactor in the presence of the catalyst and the activator to produce a poly alpha-olefin (PAO). The catalyst and activator do not contact one another outside of the oligomerization reactor.

The present application claims priority to and the benefit of U.S. Provisional Application No. 63/503,554 filed on 22 May 2023, which are hereby incorporated by reference in their entirety.

### FIELD

Embodiments of the present invention generally relate to processes for making alpha-olefin oligomers and poly alpha-olefins made therefrom.

### BACKGROUND

Alpha-olefins and poly alpha-olefins (PAOs) are commonly used as intermediates in the manufacture of many commercial products such as lubricant base oil components, basestocks, and surfactants. PAOs have a wide availability of viscosity grades, the vast majority of commercial low viscosity PAOs (below 10 cSt KV100). PAOs are typically produced from BF₃ or metallocene catalyst systems. More recently, PAOs called “hybrid PAO” have been made using both types of catalyst systems.

A hybrid PAO is a reaction product of a metallocene based intermediate (for example, an unhydrogenated metallocene dimer) with a linear alpha-olefin (LAO) using a different type of catalyst system, e.g., BF₃-alcohol promoter catalyst system. Typically, the metallocene based intermediate product from a first oligomerization reactor made from a first type of catalyst system, e.g. a metallocene, is fed to a second oligomerization reactor that uses a second type of catalyst system, e.g. BF₃-alcohol promoter catalyst system.

Conventional metallocene catalyst systems require activation and in some cases the presence of hydrogen. Metallocene catalysts are typically mixed with one or more activators in solution to activate the metallocene prior to being fed to the reactor. It is known that activated metallocene catalysts are more vulnerable to poisons than their non-activated counterparts. So when the activated catalyst batch is not used right away and held in storage, the potential for poisoning increases exponentially. Besides the potential for poisoning, when given enough time, the cation in the activator can insert into the active sites of the metallocene catalyst and essentially block the olefins to be oligomerized.

Therefore, there is a need for new processes to produce PAOs from metallocenes at high catalyst efficiency, good kinetics, and high conversions. There is also a need for improved processes and apparatus for producing PAOs, such as low viscosity PAOs including hybrid trimers, from feedstocks containing the PAO dimers.

### SUMMARY

Processes for making poly alpha-olefins (PAO) at high catalyst efficiency, good kinetics, and high conversions are provided. In at least one embodiment, the process comprises feeding at least one catalyst to an oligomerization reactor; feeding at least one activator to the oligomerization reactor; feeding one or more linear alpha olefins to the oligomerization reactor; and oligomerizing the one or more linear alpha olefins within the oligomerization reactor in the presence of the catalyst and the activator to produce a poly alpha-olefin (PAO), wherein the catalyst and activator do not contact one another outside of the oligomerization reactor.

In at least another embodiment, the process comprises feeding a first catalyst to a first oligomerization reactor; feeding an activator to the first oligomerization reactor; feeding one or more linear alpha olefin to the first oligomerization reactor; oligomerizing the one or more linear alpha olefins within the first oligomerization reactor in the presence of the first catalyst and the activator to produce a poly alpha-olefin (PAO) intermediate, wherein the first catalyst and the activator do not contact one another outside of the first oligomerization reactor; feeding the PAO intermediate and one or more additional linear alpha olefins to a second oligomerization reactor; feeding a second catalyst to the second oligomerization reactor; and oligomerizing the PAO intermediate and the one or more additional linear alpha olefins within the second oligomerization reactor in the presence of the second catalyst to produce a poly alpha-olefin (PAO) product.

In at least another embodiment, the process comprises feeding a metallocene catalyst to a first oligomerization reactor; feeding an activator to the first oligomerization reactor; feeding one or more linear alpha olefin to the first oligomerization reactor; oligomerizing the one or more linear alpha olefins within the first oligomerization reactor in the presence of the metallocene catalyst and the activator to produce a poly alpha-olefin (PAO) intermediate, wherein the metallocene catalyst and activator do not contact one another outside of the first oligomerization reactor; feeding the PAO intermediate and one or more additional linear alpha olefins to a second oligomerization reactor; feeding a BF3 catalyst to the second oligomerization reactor; and oligomerizing the PAO intermediate and the one or more additional linear alpha olefins within the second oligomerization reactor in the presence of the BF3 catalyst to produce a poly alpha-olefin (PAO) product.

These and other features and attributes of the disclosed process of the present disclosure and their advantageous applications and/or uses will be apparent from the detailed description which follows.

### BRIEF DESCRIPTION OF THE DRAWINGS

To assist those of ordinary skill in the relevant art in making and using the subject matter hereof, reference is made to the appended drawings, wherein:

FIG. 1 shows an illustrative process schematic diagram for forming poly alpha-olefins according to one or more embodiments provided herein.

FIG. 2 shows an illustrative system for making poly alpha-olefins using two or more oligomerization systems arranged in series, according to one or more embodiments provided herein.

### DETAILED DESCRIPTION

It is to be understood that the following disclosure describes several exemplary embodiments for implementing different features, structures, and/or functions of the invention. Exemplary embodiments of components, arrangements, and configurations are described below to simplify the present disclosure; however, these exemplary embodiments are provided merely as examples and are not intended to limit the scope of the invention. Additionally, the present disclosure may repeat reference numerals and/or letters in the various exemplary embodiments and across the Figures provided herein. This repetition is for the purpose of simplicity and clarity and does not in itself dictate a relationship between the various exemplary embodiments and/or configurations discussed in the Figures. Moreover, the exemplary embodiments presented below can be combined in any combination of ways, i.e., any element from one exemplary embodiment can be used in any other exemplary embodiment, without departing from the scope of the disclosure.

The present disclosure provides processes for producing poly alpha-olefins using metallocene catalyst compounds at high catalyst efficiency, excellent kinetics, and high conversions. The metallocene catalyst remains free of activation (i.e., remains unactivated) until the metallocene is added to the reactor. By only having the metallocene and activator come into contact in the reactor, the possibility of poisoning or otherwise deactivating the metallocene is significantly reduced, if not eliminated. Further, it has been surprising discovered that metallocene catalysts can be sufficiently activated within the reactor in the presence of the olefins to be oligomerized, or in the inlet piping just prior to the reactor containing the olefins to be oligomerized without any adverse effect to catalyst productivity, catalyst activity, residence time, nor the formation and selectivity of the intended PAO product. In this way, the activated catalyst avoids poisoning and maintains its reactivity for available olefins. Additionally, the inactivated catalyst is more stable than its activated counterpart, and as such. the inactivated metallocene catalyst can sit in solution for longer without degradation in quality.

“Catalyst productivity” is the quantity of PAO produced per quantity of the metallocene compound used, reported in units of gram PAO/gram metallocene). For calculating catalyst productivity, only the weight of the transition metal component of the catalyst is used.

Unless otherwise indicated, “catalyst activity” is a measure of how active the catalyst is and is reported as the mass of product PAO (P) produced per mole of catalyst (cat) used (typically reported as kg P/mol catalyst in a continuous process or kg P/mmol/hr in a batch process). For calculating catalyst activity, the molar amount of the transition metal component of the catalyst is used.

“Residence time” is defined to be the average time the reactants and products are in the reactor in a continuous process under steady-state conditions. Residence time is measured as the amount of material in the reactor (i.e. grams), divided by the outflow (i.e. grams/hr).

The term “continuous” means a system that operates without interruption or cessation for a period of time, such as where reactants are continually fed into a reaction zone and products are continually or regularly withdrawn without stopping the reaction in the reaction zone. For example, a continuous process to produce a polymer would be one where the reactants are continually introduced into one or more reactors and polymer product is continually withdrawn.

A “solution oligomerization” means an oligomerization process in which the oligomerization is conducted in a liquid medium, such as an inert solvent or monomer(s) or their blends. A solution oligomerization is typically homogeneous. A homogeneous oligomerization is one where the oligomer product is dissolved in the oligomerization medium. Such systems are typically not turbid as described in Oliveira, J. V. et al. (2000) “High-Pressure Phase Equilibria for Polypropylene-Hydrocarbon Systems,” Ind. Eng. Chem. Res., v. 39(12), pp. 4627-4633.

A bulk oligomerization means an oligomerization process in which the monomers and/or comonomers being oligomerized are used as a solvent or diluent using little or no inert solvent or diluent. A small fraction of inert solvent might be used as a carrier for catalyst and scavenger. A bulk oligomerization system contains less than about 25 wt % of inert solvent or diluent, such as less than about 10 wt %, such as less than about 1 wt %, such as 0 wt %.

As used herein, “degree of oligomerization” refers to the number of monomeric units of an oligomer. For example, an oligomer having a degree of oligomerization of 3 is an oligomer that is the reaction product of 3 monomers. A “dimer” has a degree of oligomerization of 2, and a “trimer” has a degree of oligomerization of 3.

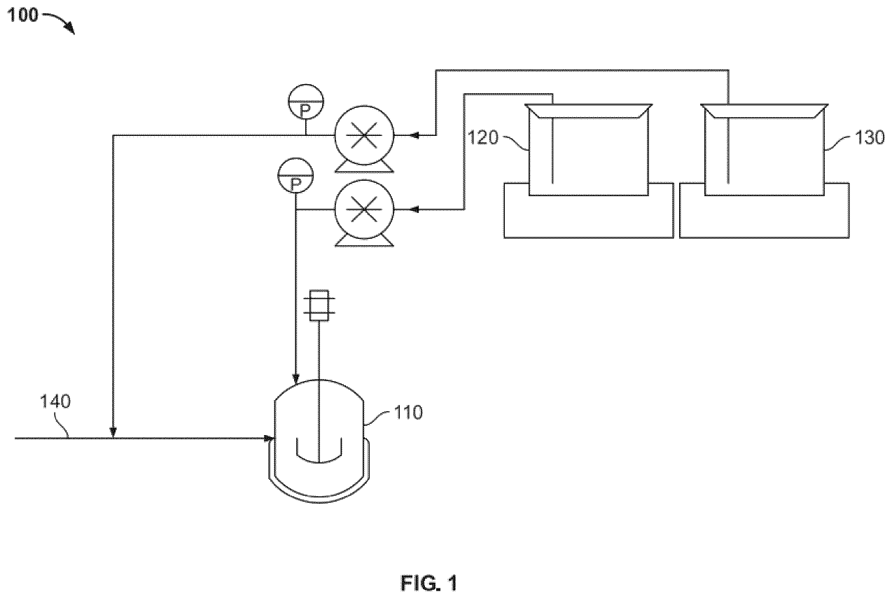

FIG. 1 depicts an illustrative system for forming poly alpha-olefins (“PAOs”) according to at least one embodiment provided herein. The system 100 includes one or more reactors 110, one or more catalyst tanks 120 and one or more activator tanks 130. The contents of the catalyst tank(s) 120 are pumped or otherwise transferred directly to the reactor 110. The contents of the activator tank(s) 130 are pumped or otherwise transferred directly to the reactor 110 or the activator is mixed with the inlet olefin feed (line 140) prior to the olefin being fed to the reactor 110. The catalyst and the activator are not contacted or otherwise mixed with one another outside of the reactor 110. By separating the catalyst from the activator until oligomerization is desired, the catalyst does not contact the activator outside of the reactor 110 and significantly reduces any chance of premature poisoning or deactivation. It has been surprisingly discovered that the catalyst can be sufficiently activated within the reactor 110 and does not require advanced activation prior to being added to the reactor 110. It has also been surprisingly discovered that there are no adverse effects to catalyst productivity, catalyst activity, residence time, nor the formation and selectivity of the intended PAO product, with this separated feed approach.

The catalyst can be any suitable metallocene catalyst. In one or more embodiments, the metallocene compound can be represented by the formula:

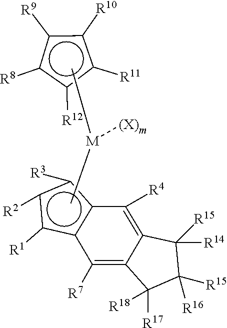

wherein: each R1, R2, and R3 is independently hydrogen or a substituted or unsubstituted linear, branched linear, or cyclic C1-C20 hydrocarbyl or silylcarbyl group; each of R4 and R7 is independently a substituted or unsubstituted linear, branched linear, or cyclic C1-C30 hydrocarbyl or silylcarbyl group; each of R8, R9, R10, R11, and R12 is independently a hydrogen, or a substituted or unsubstituted linear, branched linear, or cyclic C1-C20 hydrocarbyl, silylcarbyl, or germanyl group, or optionally at least three of R8, R9, R10, R11, and R12 are not hydrogen; each of R13, R14, R15, R16, R17, and R18 is independently hydrogen or a substituted or unsubstituted linear, branched linear, or cyclic C1-C20 hydrocarbyl or silylcarbyl group; M is a group 3, 4 or 5 transition metal; each X is independently a halogen, a hydride, an amide, an alkoxide, a sulfide, a phosphide, a diene, an amine, a phosphine, an ether, or a C1-C20 substituted or unsubstituted linear, branched, or cyclic hydrocarbyl group, or optionally two or more X moieties may together form a fused ring or ring system; and m is an integer equal to 1, 2, or 3.

Any suitable activator that is compatible with the foregoing metallocenes can be used. For example, in one or more embodiments, the activator can be or can include any one or more of the following:
N,N-dimethylanilinium tetrakis(perfluorophenyl)borate,
N,N-dimethylanilinium tetrakis(perfluoro-naphthyl)borate,
triphenylcarbonium tetrakis(perfluorophenyl)borate,
triphenylcarbonium tetrakis(perfluoronaphthyl)borate,
N,N-dimethylanilinium tetrakis(perfluorophenyl)aluminate,
N,N-dimethylanilinium tetrakis(perfluoronaphthyl)aluminate,
alumoxane,
a modified alumoxane, and
an aluminum alkyl represented by the formula (V):

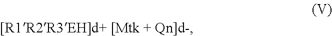

where E is nitrogen or phosphorous; d is 1, 2 or 3; k is 1, 2, or 3; n is 1, 2, 3, 4, 5, or 6; n−k=d; R1′, R2′, and R3′ are independently C1 to C50 hydrocarbyl group optionally substituted with one or more alkoxy groups, silyl groups, a halogen atoms, or halogen containing groups, w herein R1′, R2′, and R3′ together comprise 15 or more carbon atoms; Mt is an element selected from group 13 of the Periodic Table of the Elements; and each Q is independently a hydride, bridged or unbridged dialkylamido, halide, alkoxide, aryloxide, hydrocarbyl, substituted hydrocarbyl, halocarbyl, substituted halocarbyl, or halosubstituted-hydrocarbyl radical.

Examples of other suitable metallocenes, activators and scavengers are disclosed and described in US Publication No. 2021/0122859A1. U.S. Pat. No. 9,409,834 (e.g., at line 37, column 33 to line 61, column 34) provides another detailed description of suitable scavengers. Suitable scavengers can further include those mentioned in U.S. Pat. No. 5,241,025; EP-A 0426638; and WO 1997/022635.

Still referring to FIG. 1, the feed to the reactor 110 includes one or more alpha-olefins. The one or more alpha-olefins in the feed can be any one or more C2-C32 alpha-olefins, such as C4-C32 alpha-olefins, such as C6-C30 alpha-olefins, such as C6-C24 alpha-olefins, such as C6-C18 alpha-olefins, C8-C18 alpha-olefins, C6 to C16 alpha-olefins, C6-C12 alpha-olefins, or a combination thereof. Non-limiting examples of linear alpha-olefins include 1-butene, 1-pentene, 1-hexene, 1-heptene, 1-octene, 1-nonene, 1-decene, 1-undecene, 1-dodecene, 1-tridecene, 1-tetradecene, 1-pentadecene, 1-hexadecene, and a combination thereof. The alpha-olefins used herein can be produced directly from an ethylene growth process as practiced by several commercial production processes, or the alpha-olefins can be produced from Fischer-Tropsch hydrocarbon synthesis from CO/H₂ syngas. Suitable alpha-olefins can also be formed from the metathesis of internal olefins with ethylene, or from cracking of petroleum or Fischer-Tropsch synthetic wax at high temperature, or any other alpha-olefin synthesis routes. The feed olefins also can be any mixture of olefins produced from other linear alpha-olefin process containing C4 to C20 alpha-olefins as described in Chapter 3 “Routes to Alpha-Olefins” of the book Alpha Olefins Applications Handbook, Edited by G. R. Lappin and J. D. Sauer, published by Marcel Dekker, Inc. N.Y. 1989.

In some embodiments, at least a portion (e.g., at least about 80 mol %, at least about 85 mol %, at least about 90 mol %, at least about 95 mol %, at least about 96 mol %, at least about 98 mol %, at least about 99 mol %, at least about 99.5 mol %, or completely all, allowing for some impurities present in feed components) of the alpha-olefins in the feed are linear alpha-olefins (LAOs), i.e., those without a branch attached to the carbon backbone thereof. Non-limiting examples of LAOs are 1-butene, 1-pentene, 1-hexene, 1-heptene, 1-octene, 1-nonene, 1-decene, 1-undecene, 1-dodecene, 1-tridecene, 1-tetradecene, 1-pentadecene, 1-hexadecene, 1-octadecene, 1-icocene, C22, C24, C26, C28, C30 and C32 LAOs, and a combination thereof.

Where a single alpha-olefin is fed to the oligomerization reactor 110, the thus obtained PAO is a homopolymer. Homopolymers can have substantially uniform molecular structure, and accordingly desirable physical and rheological properties such as viscosity index. A homopolymer can tend to have pendant groups attached to the carbon backbone with highly uniform length.

In certain situations, a mixture of two, three, or even more alpha-olefins in the feed may be desired to produce a copolymer PAO product. To that end, alpha-olefins with the following combinations can be advantageous: C6/C8, C6/C10, C6/C12, C6/C14, C6/C16, C8/C10, C8/C12, C8/C14, C8/C16, C10/C12, C10/C14, C10/C16, C10/C18, C12/C14, C12/C16, C12/C18, C12/C20, C6/C8/C10, C6/C8/C12, C6/C8/C14, C6/C10/C12, C6/C10/C14, C8/C10/C12, C8/C11/C14, C8/C12/C14, C10/C12/C16, C10/C12/C18, C10/C14/C16, C10/C14/C18, and the like. In some embodiments, at least one of the alpha-olefins in the mixture feed can be an LAO. In some embodiments, substantially all of the alpha-olefins in the mixture feed can be LAOs.

In some embodiments, alpha-olefin monomers are mono-olefins containing one C═C bond per monomer molecule, though those olefins containing two or more C═C bonds per monomer molecule can be used as well.

In some embodiments, monomers useful herein include substituted or unsubstituted C6 to C32 alpha-olefins, or C6 to C20 alpha-olefins, or C6 to C14 alpha-olefins, or hexene, heptene, octene, nonene, decene, undecene, dodecene, tetradecene and isomers thereof. In some embodiments, the poly alpha-olefin prepared herein comprises about 50 mol % or more (such as about 60 mol % or more, such as about 70 mol % or more, such as about 80 mol % or more, such as about 90 mol % or more, such as about 99 mol % or more) of one or more C6 to C32 (such as C6 to C20, such as C8 to C18) alpha-olefin monomers.

Useful C6 to C32 alpha-olefin monomers include hexene, heptane, octene, nonene, decene, undecene, dodecene, tetradecene, substituted derivatives thereof, and isomers thereof. In some embodiments, the monomers are C6 to C20 alpha-olefins, or C6 to C14 alpha-olefins, and/or C8 to C12 alpha-olefins. In some embodiments, the olefin monomers are one (alternately two, alternately three) or more of hexene, heptene, octene, nonene, decene, dodecene, and tetradecene.

The PAOs described herein can be produced in one or more homogeneous solution processes. In each process, two or more reactors 110 in series or in parallel can be used (only one is depicted in FIG. 1). Each reactor 110 can be a continuous stirred tank reactor or plug flow reactor. Each reactor 110 may or may not have internal cooling and the monomer feed may or may not be refrigerated. See the general disclosure of U.S. Pat. No. 5,705,577 for general process conditions. The metallocene compound, activator and when required, co-activator, are delivered separately as a solution or slurry in a solvent or in the alpha-olefin feed stream. The catalyst can be supported or not supported. The catalyst and its activator(s) are not mixed outside of the reactors 110 or pre-activated and pumped as an activated solution or slurry to the reactor.

When a solid supported catalyst is used, a slurry oligomerization process generally operates in the similar temperature, pressure, and residence time range as described for a solution process. In a slurry process, a suspension of solid catalyst, promoters, monomer and comonomers are added. The suspension including diluent is intermittently or continuously removed from the reactor. The catalyst is then separated from the product by filtration, centrifuge, or settlement. The fluid is then distilled to remove solvent, any unreacted components and light product. A portion or all the solvent and unreacted component or light components can be recycled for reuse.

If the catalyst used is un-supported or is a solution catalyst, when the reaction is complete or when the product is withdrawn from the reactor 110, the product may still contain soluble, suspended, or mixed catalyst system components. These components can be deactivated and/or removed. Any of the usual catalyst deactivation methods or aqueous wash methods can be used to remove the catalyst system component.

Suitable hydrocarbon solvents are selected from C4 to C10 linear, branched or cyclic alkanes. Suitable hydrocarbon solvents also can be selected from one or more C6 to C32 alpha olefins (alternatively C8 to C16). The solvent also can be selected from one or more C6 to C32 alpha olefins (LAO) mixed with C4 to C10 linear, branched or cyclic alkanes (HC).

The oligomerization reaction mixture can then be quenched, e.g., by the addition of a quenching agent such as water, CO₂, methanol, ethanol, mixtures thereof, and the like. Subsequently, the oligomerization reaction mixture can be separated to remove the residual monomer, which can be recycled to the oligomerization reactor. Monomer removal can be carried out by means such as flashing under vacuum, distillation, or extraction. The resultant mixture can include vinylidenes, tri-substituted vinylenes, optionally di-substituted vinylenes, and optionally vinyls.

The oligomerization conditions within the reactor can include a reactor temperature of from about 0° C. to about 300° C., such as from about 10° C. to about 230° C., such as from about 25° C. to about 200° C., such as from about 100° C. to about 160° C., such as from about 110° C. to about 155° C., such as from about 130° C. to about 148° C., such as from about 135° C. to about 145° C. In some embodiments, the reactor conditions can include a reactor temperature of about 110° C., of about 130° C., about 131° C., about 132° C., about 133° C., about 134° C., about 135° C., about 136° C., about 137° C., about 138° C., about 139° C., about 140° C., about 141° C., about 142° C., about 143° C., about 144° C., about 145° C., about 146° C., about 147° C., or about 148° C. In at least one embodiment, the oligomerization conditions within the reactor can include a reactor temperature of about 110° C. or more, about 120° C. or more, such as from about 110° C. to about 180° C., such as from about 120° C. to about 180° C., such as from about 130° C. to about 180° C.

The oligomerization conditions within the reactor can include a reactor pressure of from about 1.5 psia to about 1500 psia, such as from about 7 psia to about 1200 psia, such as from about 15 psia to about 750 psia, such as from about 30 psia to about 100 psia.

The oligomerization conditions within the reactor can include a residence time such as less than about 72 hours, such as from about 1 minute to about 20 hours, such as from about 5 minutes to about 10 hours, such as from about 30 minutes to about 9 hours, such as from about 1 hours to about 5 hours, such as from about 3 hours to about 4 hours. In at least one embodiment, the reactor conditions can include a residence time of about 24 hours or less, such as about 10 hours or less, such as about 5 hours or less, such as about 3 hours or less.

Additional oligomerization conditions, such as solvent loading, catalyst loading/catalyst amount, flow rate of the catalyst system, flow rate of the alpha-olefin feed, a mol ratio of catalyst:activator, catalyst loading scavenger loading, activator loading, etc, are disclosed and described in US US20210122859A1.

The reactor effluent (e.g. PAO) can be a homopolymer made from a single alpha-olefin monomer or a copolymer made from a combination of two or more alpha-olefin monomers. In some embodiments, the alpha-olefin monomer(s) can include, can consist essentially of, or can consist of 1-hexene, 1-octene, 1-decene, 1-dodecene, or a combination thereof, such as 1-octene, 1-decene, and 1-dodecene. In an embodiment, the PAO from the first oligomerization process is a homopolymer of any C8 to C12 alpha-olefin, i.e., the PAO is a homopolymer of 1-hexene, 1-heptene, 1-octene, 1-nonene, 1-decene, 1-undecene, 1-dodecene or 1-tetradecene. In some embodiments, the PAO is a homopolymer of decene. In at least one embodiment, the PAO is a copolymer comprising decene and one or more of any of the monomers listed above.

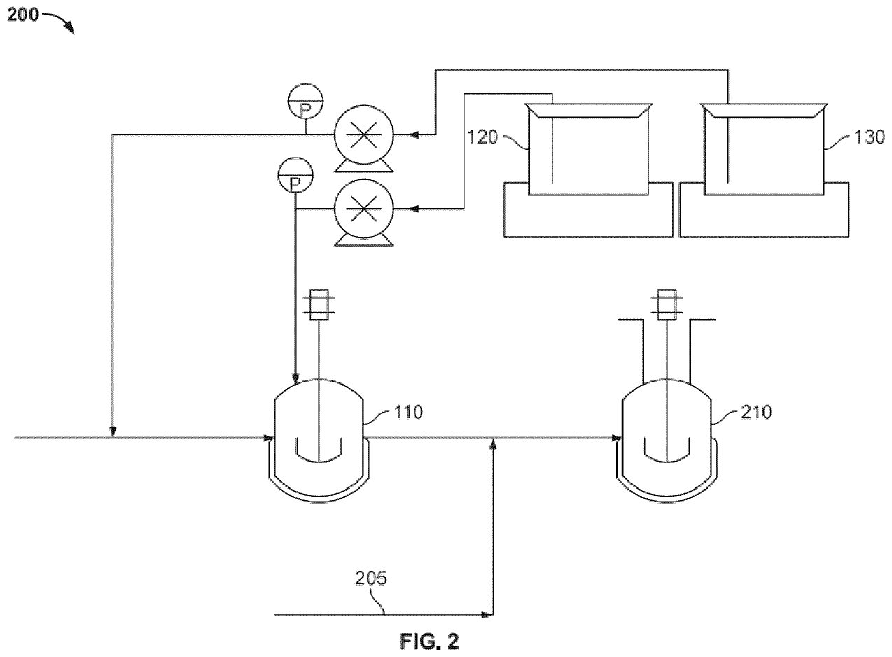

FIG. 2 shows another illustrative system 200 for making PAOs from two or more oligomerization processes. Such PAOs can be made from two or more oligomerization processes that are arranged in series, as depicted in FIG. 2. For example, effluent from a first oligomerization reactor 110 can serve solely as a feed to a second oligomerization reactor 210 or can be combined with an alpha-olefin feedstock, typically of the type used as the olefin starting material for the first oligomerization process, as described above. Other portions of the effluent from the first oligomerization reactor 110 can also be used as feedstock to the second oligomerization reactor 210, including unreacted LAO. Optionally, one or more scavengers can be added to the effluent from the first oligomerization reactor 110 via line 205. Alpha-olefins with the same attributes as those used for the first oligomerization process can be used for the second oligomerization process. For example, 100% dimer in the effluent from the first oligomerization reactor 110 (also referred to as “intermediate PAO”) is preferred, however typical ratios for the PAO dimer portion of the intermediate PAO to the alpha-olefins fraction of the intermediate PAO can be from about 99:1 to 10:90, alternately 90:10 to about 10:90, such as from about 95:5 to about 50:50 by weight of the intermediate PAO. In at least one embodiment, the PAO dimer of the intermediate PAO can make up about 50 mol % of the olefinic feed material to the second reactor since the properties and distribution of the final product, dependent in part upon the starting material, can be favorably affected by feeding the intermediate PAO at an equimolar ratio with the alpha-olefins. The olefinic feed material to the second reactor can also contain up to about 50 mol % fresh LAO.

The second oligomerization can be carried out in one or more reactors 210 such as a CSTR. In some embodiments, the residence time in the reactor 210 for the second oligomerization can be from about 1 minute to 10 hours, such as from about 1 hour to about 7 hours, such as from about 1 hour to about 2 hours. The second oligomerization also can be carried out in two reactors 210 in series, such as two continuous stirred tank reactors (CSTRs) in series. In some embodiments, the residence time in the first reactor of the second oligomerization can be from about 0.25 hour to about 5 hours, such as from about 0.5 hour to about 3 hours, and the residence time in the second reactor of the second oligomerization can be from about 0.25 hour to about 5 hours, such as from about 0.5 hour to about 3 hours.

Preferably, an acid catalyst composition or non-transition metal catalyst is used for the second oligomerization process. Such suitable catalysts can be a Lewis acid catalyst. US Patent Publication Nos. 2009/0156874 and 2009/0240012 describe a process that can be used for the second oligomerization, to which reference is made for details of feedstocks, compositions, catalysts and co-catalysts, and process conditions. The Lewis acid catalysts of US 2009/0156874 and US 2009/0240012 include the metal and metalloid halides conventionally used as Friedel-Crafts catalysts, and examples include AlCl₃, BF₃, AlBr₃, TiCl₃, and TiCl₄ either alone or with a protic promoter/activator. Boron trifluoride (BF₃) is commonly used but not particularly suitable unless it is used with a protic promoter. Useful co-catalysts are well known and described in detail in US 2009/0156874 and US 2009/0240012. Solid Lewis acid catalysts, such as synthetic or natural zeolites, acid clays, polymeric acidic resins, amorphous solid catalysts such as silica-alumina, and heteropoly acids such as the tungsten zirconates, tungsten molybdates, tungsten vanadates, phosphotungstates and molybdotungstovanadogermanates (e.g., WOx/ZrO2, WOx/MoO3) can also be used although these are not generally as favored economically. Additional process conditions and other details are described in detail in US 2009/0156874 and US 2009/0240012, and incorporated herein by reference.

Temperatures for the second oligomerization in the second reactor(s) 210 can range from about 0° C. to about 60° C., such as from about 10° C. to about 55° C., such as from about 20° C. to about 40° C., from about 10° C. to about 40° C., or from about 15° C. to about 25° C. The temperatures for the second oligomerization in the second reactor can be less than about 32° C., such as from about 15° C. to about 30° C., such as from about 20° C. to about 25° C. Reactor pressure can range from about 10 psia to about 35 psia, such as from about 15 psia to about 25 psia, such as from about 19 psia to about 21 psia.

The acid catalyst composition loading for the second oligomerization in the second reactor(s) 210 can range from about 0.5 mmol per 100 g LAO (mmolCat/100 gLAO) to about 30 mmolCat/100 gLAO, such as from about 5 mmolCat/100 gLAO to about 15 mmolCat/100 gLAO, such as from about 6 mmolCat/100 gLAO to about 14 mmolCat/100 gLAO, such as about 8 mmolCat/100 gLAO, about 10 mmolCat/100 gLAO, or about 12 mmolCat/100 gLAO.

A molar ratio of the PAO dimer of the intermediate PAO to LAO for the second oligomerization process can be about 1:1 or greater, such as from about 1.5 to about 10:1, such as from about 2:1 to about 5:1, such as from about 3:1 to about 4:1. A molar ratio of the PAO dimer of the intermediate PAO to LAO for the second oligomerization process can be from about 0.1:1 to about 10:1, such as from about 0.5 to about 5:1, such as from about 0.5:1 to about 3:1, such as from about 0.8:1 to about 1.2:1, such as from about 0.9:1 to about 1.1:1.

The effluent from the second reactor(s) 210 in the second oligomerization process can be a homopolymer made from a single alpha-olefin monomer or a copolymer made from a combination of two or more alpha-olefin monomers. The alpha-olefin monomer(s) can include, consist essentially of, or consist of 1-hexene, 1-octene, 1-decene, 1-dodecene, or a combination thereof, such as 1-octene, 1-decene, and 1-dodecene. The PAO from the second oligomerization process can be a homopolymer of any C8 to C12 alpha-olefin, i.e., the PAO is a homopolymer of 1-hexene, 1-heptene, 1-octene, 1-nonene, 1-decene, 1-undecene, 1-dodecene or 1-tetradecene. In some embodiments, the PAO is a homopolymer of decene. In at least one embodiment, the PAO is a copolymer comprising decene and one or more of any of the monomers listed above.

### Functionalized Paos and Uses of Functionalized Paos

The intermediate PAO from the first oligomerization process and/or the PAO from the second oligomerization process (the “PAO product”) can be functionalized with one or more reactants (and can be optionally hydrogenated) through various chemical reactions to produce a functionalized PAO product, as is known in the art, and as described in U.S. Pat. No. 6,022,929; A. Toyota et al. (2002) Polymer Bulletin, v. 48, pp. 213-219; and J. Am. Chem. Soc. (1990) v. 112, pp. 7433-7434. In some embodiments the functionalized PAO's produced herein are further functionalized (derivatized), such as described in U.S. Pat. No. 6,022,929; A. Toyota et al. (2002) Polymer Bulletin, v. 48, pp. 213-219; and J. Am. Chem. Soc. (1990) v. 112, pp. 7433-7434; and WO 2009/155472.

An unsaturated PAO product provided herein can contain greater than or equal to about 80 mol % vinylidenes, such as 90 mol % vinylidenes, such as 93 mol % vinylidenes, based on total moles of vinyls, vinylidenes, di-substituted vinylenes, and tri-substituted vinylenes contained therein. In some embodiments, the unsaturated poly alpha-olefin product comprises 93 mol % to 99.9 mol % of vinylidenes; 0.1 mol % to 3.5 mol % of tri-substituted vinylenes; 3.0 mol % or less of di-substituted vinylenes; 3.0 mol % or less of vinyl groups; based on total moles of vinylidenes, tri-substituted vinylenes, di-substituted vinylenes, and vinylidenes contained therein; and a number average molecular weight (Mn) of 1500 g/mol or less as measured by 1H NMR.

In some embodiments, the unsaturated PAO product can contain less than or equal to about 1.0 mol % di-substituted vinylenes, when present; less than or equal to about 1.0 mol % vinyl groups when present; and a number average molecular weight (Mn) of 1000 g/mol or less as measured by 1H NMR.

The resulting PAO can contain two or more monomers, or three or more monomers, or four or more monomers, or five or more monomers. For example, a C8, C10, C12-linear alpha-olefin mixture, or a C6, C7, C8, C9, C10, C11, C12, C13, C14-linear alpha-olefin mixture, or a C6, C8, C10, C12, C14, C16, C18-linear alpha-olefin mixture can be used as a feed. The resulting PAO can be less than about 50 mol % of C2, C3, and C4 monomers, or less than about 40 mol %, or less than about 30 mol %, or less than about 20 mol %, or less than about 10 mol %, or less than about 5 mol %, or less than about 3 mol %, or about 0 mol %. The resulting PAO can be less than about 50 mol % of ethylene, propylene and butene, or less than about 40 mol %, or less than about 30 mol %, or less than about 20 mol %, or less than about 10 mol %, or less than about 5 mol %, or less than about 3 mol %, or about 0 mol %. The resulting PAO can be less than about 40 mol %, or less than about 20 mol %, or less than about 10 mol %, or less than about 5 mol %, or less than about 3 mol %, or about 0 mol % of ethylene.

The polyalphaolefin (PAO) product can be used as a base oil for lubricants and the like. The resulting PAOs can have number average molecular weights that typically vary from 250 to 3,000; or 250 to 1,000; or 300 to 700; or 400 to 500, and viscosities at 100° C. of 1.5 cSt to 150 cSt. PAO fluids of particular use may include those having KV100 of 3 cSt, 3.4 cSt, and/or 3.6 cSt, and combinations thereof. Mixtures of PAO fluids having KV100 viscosity of 1.5 to 150 cSt or more are also useful.

The poly alpha-olefin (PAO) product can have any one or more of the following properties:
a. Kinematic viscosity (KV100° C. as measured according to ASTM D445) of from 1 to 10 cSt, such as 1 cSt to 9 cSt, 2 to 8 cSt, or 3 to 6 cSt, including 2.5 cSt, 3.0 cSt, 3.5 cSt, 4.0 cSt, 5.0 cSt or 6.0 cSt;
b. Kinematic viscosity (KV40° C. according to ASTM D445): of from 3 to 20 cSt, such as 3 cSt to 18 cSt, 5 to 15 cSt, or 10 to 16 cSt, including 12.5 cSt, 13.0 cSt, 13.5 cSt, 14.0 cSt, 15.0 cSt or 16.0 cSt;
c. Viscosity index (VI as measured according to ASTM D445) of about 110 to about 150; about 115 to about 140; or about 120 to about 130, such as about 120, 125, 128, 130, or 135.
d. Pour Point (as measured by ASTM D97) of −10° C. or lower, or −20° C. or lower, or −25° C. or lower, about −50° C. or lower; −60° C. or lower, or −70° C. or lower, or −80° C. or lower, −85° C. or lower, or −90° C. or lower.
e. Noack volatility, as measured according to ASTM D 5800, of about 5% to 25%; 10% to 15%; or 11% to 14%, or about 10%, about 11%, about 12%, about 12.5%, or about 13%.

### EXAMPLES

To facilitate a better understanding of the embodiments of the present invention, the following examples of preferred or representative embodiments are given. In no way should the following examples be read to limit, or to define, the scope of the invention.

As referenced below, Catalyst C-2 and Activator A-2 are represented by the following structures:

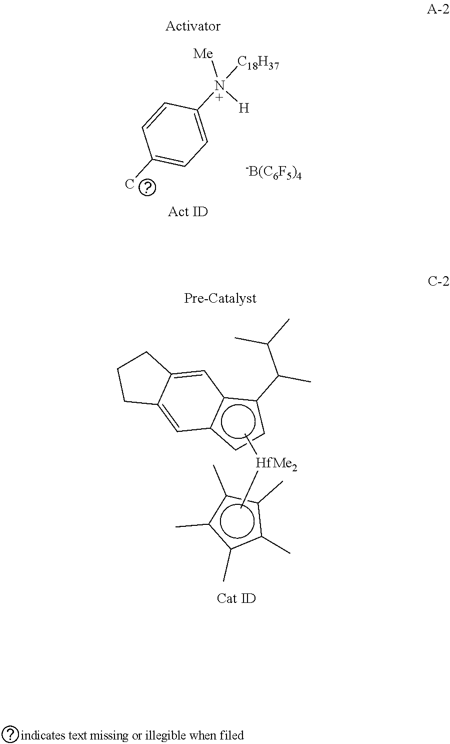

### Example 1 (Ex. 1—In Situ Reactor Catalyst Activation)

A 2 gallon pressurized Parr reactor was used to conduct a continuously stirred metallocene oligomerization reaction. The reactor had a dip tube for the catalyst solution injection as well as a feed line for various types of linear-alpha-olefins. The reactor was equipped with an external heater, as well as an internal cooling loop to control the temperature of the reaction. Catalyst batch and activator batch preparations were carried out in an N₂ purged dry box using standard air sensitive procedures.

Catalyst batch preparation: 398.6 grams of toluene were passed through a commercially available adsorbent (UOP 13× Molecular Sieve) to remove moisture then added into a clean empty jar with a magnetic stirrer on a stirring plate, 0.38 grams of tri-n-octyl aluminum was then added into the toluene to scavenge any contaminants that were not captured in the adsorbent beds. After 5 minutes of mixing, 1.0 grams of Catalyst C-2 was dissolved into the mixture and mixed for 10 minutes.

Activator batch preparation: In a different jar, 398.1 grams of toluene was passed through a commercially available adsorbent (UOP 13× Molecular Sieve) to remove moisture then added into a different clean empty jar with a magnetic stirrer on a stirring plate, 0.38 grams of tri-n-octyl aluminum was then added into the toluene to scavenge any contaminants that were not captured in the adsorbent beds. After 5 minutes of mixing, 1.52 grams of the Activator A-2 was dissolved into the mixture and mixed for 10 minutes.

The 1-decene feed was fed through two adsorbent beds in series, loaded with UOP AZ-300 and finally entered the reactor through the LAO line. The 1-decene was fed at 2080 g/hr into the Parr reactor operated under vacuum where it was initially degassed. The activated catalyst batch was connected to a piston pump, where it was fed at 11.1 g/hr (equivalent to 13 ppm of Catalyst C-2 in the reactor) and entered the reactor through the catalyst dip tube. The activator batch was also connected to a piston pump, where it was fed at 11.1 g/hr; the activator solution was mixed with the 1-decene in the LAO line. The reactor was operated liquid full (3 hour residence time), with back pressure of 25 psig and at 140° C. The reactor effluent was sent to a quench vessel, where the catalyst was quenched with water at 90° C. at the same pressure as the reactor. The quench effluent then passed through a 1 micron filter and filled the product collection pails.

### Comparative Example 1 (Comp. Ex. 1—External Catalyst Activation)

Similar to Example 1 above, a 2 gallon pressurized Parr reactor was used to conduct a continuously stirred metallocene oligomerization reaction. The reactor had a dip tube for the catalyst solution injection as well as a feed line for various types of linear-alpha-olefins. The reactor was equipped with an external heater, as well as an internal cooling loop to control the temperature of the reaction. Activated catalyst batch preparation was carried out in an N₂ purged dry box using standard air sensitive procedures.

Activated catalyst batch preparation: 396.7 grams of toluene was passed through a commercially available adsorbent (UOP 13× Molecular Sieve) to remove moisture then added into a clean empty jar with a magnetic stirrer on a stirring plate, 0.77 grams of tri-n-octyl aluminum was then added into the toluene to scavenge any contaminants that were not captured in the adsorbent beds. After 5 minutes of mixing, 1.0 grams of Catalyst C-2 was dissolved into the mixture and mixed for 10 minutes. Next, 1.52 grams of the Activator A-2 was dissolved into the catalyst mixture and mixed for 10 minutes.

The 1-decene feed was fed through two adsorbent beds in series, loaded with UOP AZ-300 and finally entered the reactor through the LAO line. The 1-decene was fed at 2080 g/hr into the Parr reactor operated under vacuum where it was initially degassed. The activated catalyst batch was connected to a piston pump, where it was fed at 10.4 g/hr (equivalent to 13 ppm of Catalyst C-2 in the reactor) and entered the reactor through the catalyst dip tube. The reactor was operated liquid full (3 hour residence time), with back pressure of 25 psig and at 140° C. The reactor effluent was sent to the quench vessel, where the catalyst was quenched with water at 90° C. at the same pressure as the reactor. The quench effluent then passed through 1 micron filter and filled the product collection pails.

The products from Example 1 and Comparative Example 1 were analyzed via GC to determine conversion and selectivity. Gas chromatography (GC) was used to determine the composition of the synthesized oligomers. The gas chromatograph used in the examples was an Agilent Technologies 7890A model equipped with a 30 meter column with an internal diameter of 0.28 mm and a flame ionization detector. A ^{~}0.2000-0.3000 g sample is diluted in methylene chloride solvent, ^{~}0.0600-0.1000 g nonane internal standard was added, and the mixture was injected into the column. The starting temperature was about 40° C., held for about 1 minute, program-heated at about 15° C. per minute to about 250° C. and held for about 2 minutes. The sample is then heated at a rate of about 25° C. per minute to about 360° C. and held for about 17.3 minutes. The oligomer distribution was determined by the GC method. Results are shown in Table 1.

As shown in Table 1, the in situ reactor activation (Ex. 1) presented an improvement over the typical external activation (Comp. Ex. 1). An increase in dimer selectivity and conversion was noticeable, leading to an increase in dimer yield.

TABLE 1
Product summary / Results

| Experiment | EX. 1 | COMP EX. 1 |
|---|---|---|
| Activation site | In situ Reactor | External (catalyst vessel) |
| Alpha-conversion | 84.49% | 80.55% |
| Non α-c10 | 4.05% | 3.93% |
| Dimer yield | 74.81% | 70.27% |
| Trimer yield | 5.66% | 6.32% |
| Heavies yield | 0.52% | 0.73% |

### Example 2 (EX. 2—in Reactor Activation—Hybrid PAO)

Example 1 was replicated and the reactor effluent from the oligomerization was distilled to obtain unreacted monomers and dimers. Additional 1-decene was added to the foregoing mixture, to generate 1:1 mol ratio of monomer to dimer mixture.

Two 2 liter pressurized Parr reactors (Reactor 2A and Reactor 2B) were used to conduct step 2 of the oligomerization. Reactor 2A had an inlet for various types of linear-alpha-olefins as well as a dip tube for the catalyst system. The Reactor 2B was connected to Reactor 2A to drive the conversion further. The catalyst system used was butanol/butyl acetate in a molar ratio of about 1:1, saturated with BF₃ at atmospheric temperature and pressure. This catalyst was made by charging the butanol and butyl acetate into a 5 L round bottom flask and then bubbling BF₃ into the mixture through a dip tube.

The feed mixture was fed at 600 g/hr into Reactor 2A that was operated under vacuum and initially degassed. The catalyst mixture was fed at a ratio of about 15 mmol/100 g olefin which equals to 14.7 g/hr. Reactor 2A was held at 1350 ml level, and reactor 2B was held at 600 ml level (1.75 hours residence time in reactor 12A and 0.78 hours in Reactor 2B). The Reactor 2B was held at 5 psig of BF₃ pressure. A 1:1 mole ratio of butanol/butyl acetate mixture was then added to the reactor 2B effluent to capture any free BF₃. The effluent was then sent to the quench reactor where the acidic catalyst was quenched with 10% caustic. The resultant sample was water washed multiple times and the oil phase analyzed by GC.

### Comparative Example 2 (COMP EX. 2—External Activation—Hybrid PAO)

Comparative Experiment 1 was replicated and the reactor effluent from oligomerization was distilled to obtain unreacted monomers and dimers. Additional 1-decene was added to the aforementioned mixture, to generate 1:1 mol ratio of monomer to dimer mixture.

Two 2 liter pressurized Parr reactors were used to conduct step 2 of the oligomerization. Reactor 2A had an inlet for various types of linear-alpha-olefins as well as a dip tube for the catalyst system. The second reactor (2B) was connected to reactor 2A to drive the conversion further. The catalyst system used was butanol/butyl acetate in a molar ratio of about 1:1, saturated with BF₃ at atmospheric temperature and pressure. This catalyst was made by charging the butanol and butyl acetate into a 5 L round bottom flask and then bubbling BF₃ into the mixture through a dip tube.

The feed mixture was fed at 600 g/hr into a Parr reactor operated under vacuum where it is initially degassed. The feed mixture then enters reactor 2A where it contacted the catalyst mixture which was fed at a ratio of about 15 mmol/100 g olefin which equals to 14.7 g/hr. Reactor 2A was held at 1350 ml level, and reactor 2B was held at 600 ml level (1.75 hours residence time in reactor 12A and 0.78 hours in reactor 2B). The reactor 2B was held at 5 psig of BF₃ pressure. A 1:1 mole ratio of butanol/butyl acetate mixture was then added to the reactor 2B effluent to capture any free BF₃. The effluent was then sent to the quench reactor where the acidic catalyst was quenched with 10% caustic. The resultant sample was water washed multiple times and the oil phase analyzed by GC.

The results are shown in Table 2 below. The results of the second reaction step are essentially unchanged. However, since there is an increase in conversion in the first step of reaction, this enables additional feed to the second reaction, which increases the overall yield of the trimer, the prime product.

TABLE 2
Two reactors in series

| Experiment | EX. 2 | COMP EX. 2 |
|---|---|---|
| First Reaction Activation | In reactor activation | External activation |
| Second Reactor α-Conversion | 98.8% | 98.7% |
| Second Reactor Dimer yield | 5.7% | 5.6% |
| Second Reactor Trimer yield | 76.7% | 78.5% |
| Second Reactor Heavies yield | 16.3% | 14.5% |
| Overall PAO yield on 100 g | 115.6 grams* | 111.1 grams* |
| basis feed to first step reaction | | |

*Overall PAO yield is more than 100 grams as additional LAO is fed into the second reactor. The LAO fed into the second ratio is dependent on the intermediate PAO yield from the first reactor and the desired intermediate PAO to fresh LAO ratio. This ratio is maintained at 1:1 mol ratio in all the aforementioned experiments.

Additional aspects and features of the present disclosure include any of the following numbered embodiments:

Embodiment I: A process for making a poly alpha-olefin (PAO), comprising feeding at least one catalyst to an oligomerization reactor; feeding at least one activator to the oligomerization reactor; feeding one or more linear alpha olefins to the oligomerization reactor; and oligomerizing the one or more linear alpha olefins within the oligomerization reactor in the presence of the catalyst and the activator to produce a poly alpha-olefin (PAO), wherein the catalyst and activator do not contact one another outside of the oligomerization reactor.

Embodiment II: The process of Embodiment I, wherein the at least one catalyst comprises a metallocene.

Embodiment III: The process of Embodiments I or II, wherein the one or more linear alpha olefins comprise any one or more C2-C32 alpha-olefins, C4-C32 alpha-olefins, C6-C30 alpha-olefins, C6-C24 alpha-olefins, C6-C18 alpha-olefins, C8-C18 alpha-olefins, C6 to C16 alpha-olefins, or C6-C12 alpha-olefins.

Embodiment IV: The process according to any of Embodiments I to III, wherein the one or more linear alpha olefins are selected from the group consisting of 1-butene, 1-pentene, 1-hexene, 1-heptene, 1-octene, 1-nonene, 1-decene, 1-undecene, 1-dodecene, 1-tridecene, 1-tetradecene, 1-pentadecene, and 1-hexadecene.

Embodiment V: The process according to any of Embodiments I to IV, wherein the oligomerization reactor is a CSTR.

Embodiment VI: A process for making a poly alpha-olefin (PAO), comprising: feeding a first catalyst to a first oligomerization reactor; feeding an activator to the first oligomerization reactor; feeding one or more linear alpha olefin to the first oligomerization reactor; oligomerizing the one or more linear alpha olefins within the first oligomerization reactor in the presence of the first catalyst and the activator to produce a poly alpha-olefin (PAO) intermediate, wherein the first catalyst and the activator do not contact one another outside of the first oligomerization reactor; feeding the PAO intermediate and one or more additional linear alpha olefins to a second oligomerization reactor; feeding a second catalyst to the second oligomerization reactor; and oligomerizing the PAO intermediate and the one or more additional linear alpha olefins within the second oligomerization reactor in the presence of the second catalyst to produce a poly alpha-olefin (PAO) product.

Embodiment VII: The process of Embodiment VI, wherein the first catalyst comprises a metallocene and the second catalyst comprises a Lewis acid.

Embodiment VIII: The process of Embodiments VI or VII wherein the one or more linear alpha olefins comprise any one or more C2-C32 alpha-olefins, C4-C32 alpha-olefins, C6-C30 alpha-olefins, C6-C24 alpha-olefins, C6-C18 alpha-olefins, C8-C18 alpha-olefins, C6 to C16 alpha-olefins, or C6-C12 alpha-olefins.

Embodiment IX: The process according to any of Embodiments VI to VIII, wherein the one or more linear alpha olefins are selected from the group consisting of 1-butene, 1-pentene, 1-hexene, 1-heptene, 1-octene, 1-nonene, 1-decene, 1-undecene, 1-dodecene, 1-tridecene, 1-tetradecene, 1-pentadecene, and 1-hexadecene.

Embodiment X: The process according to any of Embodiments VI to IX, wherein the one or more linear alpha olefins fed to the first reactor are different than the one or more additional linear alpha olefins fed to the second reactor.

Embodiment XI: The process according to any of Embodiments VI to X, wherein the one or more linear alpha olefins fed to the first reactor are the same as the one or more additional linear alpha olefins fed to the second reactor.

Embodiment XII: The process according to any of Embodiments VI to XI, wherein the first and second reactors are CSTR.

Embodiment XIII: The process according to any of Embodiments VI to XII, wherein the first catalyst and the first activator are each fed independently to the first oligomerization reactor.

Embodiment XIV: The process according to any of Embodiments VI to XIII, wherein the second catalyst is BF3.

Embodiment XV: A process for making a poly alpha-olefin (PAO), comprising: feeding a metallocene catalyst to a first oligomerization reactor; feeding an activator to the first oligomerization reactor; feeding one or more linear alpha olefin to the first oligomerization reactor; oligomerizing the one or more linear alpha olefins within the first oligomerization reactor in the presence of the metallocene catalyst and the activator to produce a poly alpha-olefin (PAO) intermediate, wherein the metallocene catalyst and activator do not contact one another outside of the first oligomerization reactor; feeding the PAO intermediate and one or more additional linear alpha olefins to a second oligomerization reactor; feeding a BF3 catalyst to the second oligomerization reactor; and
oligomerizing the PAO intermediate and the one or more additional linear alpha olefins within the second oligomerization reactor in the presence of the BF3 catalyst to produce a poly alpha-olefin (PAO) product.

Embodiment XVI: The process of Embodiment XV, wherein the metallocene catalyst and the activator are each fed independently to the first oligomerization reactor.

Embodiment XVII: The process of Embodiments XV or XVI, wherein the activator is mixed with the one or more linear alpha olefin and feed together to the first oligomerization reactor.

Embodiment XVIII: The process according to any of Embodiments XV to XVII, wherein the one or more linear alpha olefins fed to the first reactor are different than the one or more additional linear alpha olefins fed to the second reactor.

Embodiment XIX: The process according to any of Embodiments XV to XVIII, wherein the one or more linear alpha olefins fed to the first reactor are the same as the one or more additional linear alpha olefins fed to the second reactor.

All numerical values are “about” or “approximately” the indicated value, and take into account experimental error and variations that would be expected by a person having ordinary skill in the art.

Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A process for making a poly alpha-olefin (PAO), comprising:
feeding at least one catalyst to an oligomerization reactor;
feeding at least one activator to the oligomerization reactor;
feeding one or more linear alpha olefins to the oligomerization reactor; and
oligomerizing the one or more linear alpha olefins within the oligomerization reactor in the presence of the catalyst and the activator to produce a poly alpha-olefin (PAO), wherein the catalyst and activator do not contact one another outside of the oligomerization reactor.

2. The process of claim 1, wherein the at least one catalyst comprises a metallocene.

3. The process of claim 1, wherein the one or more linear alpha olefins comprise any one or more C2-C32 alpha-olefins, C4-C32 alpha-olefins, C6-C30 alpha-olefins, C6-C24 alpha-olefins, C6-C18 alpha-olefins, C8-C18 alpha-olefins, C6 to C16 alpha-olefins, or C6-C12 alpha-olefins.

4. The process of claim 1, wherein the one or more linear alpha olefins are selected from the group consisting of 1-butene, 1-pentene, 1-hexene, 1-heptene, 1-octene, 1-nonene, 1-decene, 1-undecene, 1-dodecene, 1-tridecene, 1-tetradecene, 1-pentadecene, and 1-hexadecene.

5. The process of claim 1, wherein the oligomerization reactor is a CSTR.

6. A process for making a poly alpha-olefin (PAO), comprising:
feeding a first catalyst to a first oligomerization reactor;
feeding an activator to the first oligomerization reactor;
feeding one or more linear alpha olefin to the first oligomerization reactor;
oligomerizing the one or more linear alpha olefins within the first oligomerization reactor in the presence of the first catalyst and the activator to produce a poly alpha-olefin (PAO) intermediate, wherein the first catalyst and the activator do not contact one another outside of the first oligomerization reactor;
feeding the PAO intermediate and one or more additional linear alpha olefins to a second oligomerization reactor;
feeding a second catalyst to the second oligomerization reactor; and
oligomerizing the PAO intermediate and the one or more additional linear alpha olefins within the second oligomerization reactor in the presence of the second catalyst to produce a poly alpha-olefin (PAO) product.

7. The process of claim 6, wherein the first catalyst comprises a metallocene and the second catalyst comprises a Lewis acid.

8. The process of claim 6, wherein the one or more linear alpha olefins comprise any one or more C2-C32 alpha-olefins, C4-C32 alpha-olefins, C6-C30 alpha-olefins, C6-C24 alpha-olefins, C6-C18 alpha-olefins, C8-C18 alpha-olefins, C6 to C16 alpha-olefins, or C6-C12 alpha-olefins.

9. The process of claim 6, wherein the one or more linear alpha olefins are selected from the group consisting of 1-butene, 1-pentene, 1-hexene, 1-heptene, 1-octene, 1-nonene, 1-decene, 1-undecene, 1-dodecene, 1-tridecene, 1-tetradecene, 1-pentadecene, and 1-hexadecene.

10. The process of claim 6, wherein the one or more linear alpha olefins fed to the first reactor are different than the one or more additional linear alpha olefins fed to the second reactor.

11. The process of claim 6, wherein the one or more linear alpha olefins fed to the first reactor are the same as the one or more additional linear alpha olefins fed to the second reactor.

12. The process of claim 6, wherein the first and second reactors are CSTR.

13. The process of claim 6, wherein the first catalyst and the first activator are each fed independently to the first oligomerization reactor.

14. The process of claim 7, wherein the second catalyst is BF₃.

15. A process for making a poly alpha-olefin (PAO), comprising:
feeding a metallocene catalyst to a first oligomerization reactor;
feeding an activator to the first oligomerization reactor;
feeding one or more linear alpha olefin to the first oligomerization reactor;
oligomerizing the one or more linear alpha olefins within the first oligomerization reactor in the presence of the metallocene catalyst and the activator to produce a poly alpha-olefin (PAO) intermediate, wherein the metallocene catalyst and activator do not contact one another outside of the first oligomerization reactor;
feeding the PAO intermediate and one or more additional linear alpha olefins to a second oligomerization reactor;
feeding a BF₃ catalyst to the second oligomerization reactor; and
oligomerizing the PAO intermediate and the one or more additional linear alpha olefins within the second oligomerization reactor in the presence of the BF₃ catalyst to produce a poly alpha-olefin (PAO) product.

16. The process of claim 15, wherein the metallocene catalyst and the activator are each fed independently to the first oligomerization reactor.

17. The process of claim 15, wherein the activator is mixed with the one or more linear alpha olefin and feed together to the first oligomerization reactor.

18. The process of claim 15, wherein the one or more linear alpha olefins fed to the first reactor are different than the one or more additional linear alpha olefins fed to the second reactor.

19. The process of claim 15, wherein the one or more linear alpha olefins fed to the first reactor are the same as the one or more additional linear alpha olefins fed to the second reactor.
